(19) United States
(12) Patent Application Publication
(10) Pub. No.: US 2017/0280998 A1
(43) Pub. Date: Oct. 5, 2017

(54) NON CONTACT EYEBALL VIBRATION TYPE TONOMETER

(71) Applicant: Adtex Inc., Gunma (JP)

(72) Inventors: Yuichi Ariga, Yamagata (JP); Sigehiko Masubuchi, Gunma (JP)

(21) Appl. No.: 15/091,087

(22) Filed: Apr. 5, 2016

(51) Int. Cl. A61B 3/16

(52) U.S. Cl. CPC A61B 3/165

(57) ABSTRACT

The problem addressed by the present invention is to provide a tonometer that is capable of measuring the intraocular pressure of a subject eye without the subject or persons around him experiencing noise, and without blowing air against the subject eye. With the present invention, a non contact eyeball vibration type tonometer is provided that includes: a parametric speaker that directs sound waves against the front surface of a subject eye; a detection device that detects data related to vibration of the subject eye caused by these sound waves from the parametric speaker; and a processing device that calculates the intraocular pressure of the subject eye from this vibration data for the subject eye.

### FIELD OF THE INVENTION

The present invention relates to a tonometer, and in particular to a non contact eyeball vibration type tonometer.

### BACKGROUND OF THE INVENTION

As disclosed in, for example, U.S. Pat. No. 3,585,849, in a prior art device for measuring intraocular pressure, a method is employed of blowing air against the subject eyeball, and detecting the resulting concavity of the subject eyeball optically.

Furthermore, in U.S. Pat. No. 5,148,807 and in publication WO03/082087, tonometers are disclosed that do not blow air against the subject eyeball, but instead vibrate it with sound.

### SUMMARY OF THE INVENTION

With the method of blowing air against the subject eyeball disclosed in U.S. Pat. No. 3,585,849, there are adverse effects due to this blowing of air, such as discomfort, blinking, possible infection, and so on. And with the method disclosed in U.S. Pat. No. 5,148,807, while there is no requirement for blowing of air against the eyeball, a loud sound is required for vibrating the subject eyeball. And with the method disclosed in WO03/082087, although a cup is used for enhancing the sound pressure, some background sound diffuses out to the surroundings; and furthermore this can hardly be termed a non-contact type method.

In consideration of the circumstances described above, the object of the present invention is to provide a tonometer that can measure the intraocular pressure in a non-contact manner, without blowing air against the subject eyeball, and without generating audio at a volume so great as to cause discomfort to the subject and also to surrounding persons.

As a result of extensive study by the present inventors, the present invention has been formulated as follows:
(1) A non contact eyeball vibration type tonometer, comprising: a parametric speaker that directs sound waves against the front surface of a subject eye; a detection device that detects vibration data of the subject eye caused by the sound waves from the aforementioned parametric speaker; and a processing device that calculates the intraocular pressure of the subject eye from the aforementioned data of the subject eye.
(2) The non contact eyeball vibration type tonometer according to (1), wherein the parametric speaker comprises a plurality of ultrasound generating elements that are arranged upon a spherical surface focused upon the front surface of the subject eye.
(3) The non contact eyeball vibration type tonometer according to (1) or (2), wherein the parametric speaker comprises an ultrasound generating element capable of generating an ultrasound signal of 30 to 50 kHz that is modulated at a frequency of 10 to 100 Hz, and wherein said element is capable of sweeping said frequency over time.
(4) The non contact eyeball vibration type tonometer according to any of (1) to (3), wherein the detection device comprises an ultrasonic detection element.
(5) The non contact eyeball vibration type tonometer according to any of (1) to (4), wherein the detection device comprises a light reception element that detects vibration of the subject eye optically.

According to the present invention, it is possible to generate sufficient sound pressure for measurement in the vicinity of the front surface of the subject eye; and moreover, since the volume of any sound reaching the subject or surrounding persons is extremely small, accordingly it is possible to vibrate the subject eye without imparting any feeling of discomfort. In concrete terms, since, according to the present invention, the eyeball is caused to vibrate by this eyeball vibration type tonometer, the characteristic frequency of the eyeball is obtained, and from this characteristic frequency the intraocular pressure, which is the pressure internal to the eyeball, is detected, accordingly it is possible to perform measurement of the intraocular pressure at high accuracy and in a less invasive and less disturbing manner, and prior art risks that were attendant upon blowing air at the eyeball are eliminated, such as discomfort to the subject, splashing around of tears (such splashing can cause hospital-acquired infection), poor eyelid opening in elderly persons (which can reduce data reliability), and so on. Furthermore, since vibration is applied only in the immediate vicinity of the subject eye and almost no vibration is applied around the eye, accordingly there is the advantageous aspect that the influence of vibration in the surroundings of the eye is very small, so that it is possible to detect the vibration of the subject eye with good accuracy.

### BRIEF DESCRIPTION OF THE DRAWINGS

FIG. 1 is a schematic drawing of a spherical surface upon which ultrasound generating elements are to be arranged;

FIG. 2 is a schematic drawing showing an example of how ultrasound generating elements can be arranged;

FIG. 3 is a figure showing the schematic layout of an example of the non contact eyeball vibration type tonometer of the present invention;

FIG. 4 is a figure showing the schematic layout of another example of the non contact eyeball vibration type tonometer of the present invention;

FIG. 5 is a schematic figure showing the system structure of an example of the non contact eyeball vibration type tonometer of the present invention; and

FIG. 6 is a schematic figure showing an example of signal timings employed in the present invention.

### DETAILED DESCRIPTION

The present invention will now be explained while referring to the drawings as appropriate. However, the present invention is not limited to the mode of implementation shown in these figures. The reference symbols in the drawings have the following meanings:

10: spherical surface

11: center

20: ultrasound generating element

21, 31, 41: subject eyes

30, 40: parametric speakers

32: ultrasound sensor

33, 44: ultrasound waves

34: vibration data

42: light source

43: light detector

45, 46: light beams

The non contact eyeball vibration type tonometer (hereinafter sometimes simply termed a “tonometer”) of the present invention employs a parametric speaker. A parametric speaker is a speaker that generates low frequency sound pressure by amplitude modulating a signal that drives one or more ultrasound generating elements at low frequency, due to non-linearity generated by the ultrasound. It is possible to obtain very sharp directivity with the use of ultrasound. For the details of the concrete structures and modes of operation of parametric speakers themselves, reference may be made to appropriate prior art documentation in the speaker field.

Typically, a parametric speaker is built from a plurality of ultrasound generating elements arranged in a predetermined configuration. Desirably, these ultrasound generating elements are made to be capable of generating ultrasound signals of 30 to 100 kHz modulated at a frequency of 5 to 100 Hz. The modulation described above is more desirably in the range of 10 to 100 Hz, and yet more desirably is in any appropriate range within the range of 10 to 100 Hz, providing that the characteristic frequency of the subject eyeball is covered by this range. The ultrasound signal is desirably in the range of from 30 to 50 kHz, and more desirably is in any appropriate range within the range of 30 to 100 kHz. Yet more desirably, the ultrasound generating elements are adapted so that the above frequency of modulation can be swept over time. According to the theory of parametric speakers, each of the ultrasound generating elements is driven at an ultrasound frequency such as that described above by way of example, and modulation is performed upon its driving wave at a frequency such as that described above by way of example. With regard to the generation of audible sound (i.e. audible sound waves) for the present invention by a parametric speaker, it should be understood that such audible sound could be generated by beat sound due to a frequency difference. In more concrete terms, in order to generate low frequency sound pressure, the group of generating elements may be divided into two subgroups, and a frequency difference may be set up between the drive frequency for one subgroup and the drive frequency for the other subgroup, so that sound pressure at low frequency is generated by the beats generated by this frequency difference. Or it would also be possible to generate low frequency sound (i.e. sound waves) by exploiting the non-linearity that occurs when vibrations become sound.

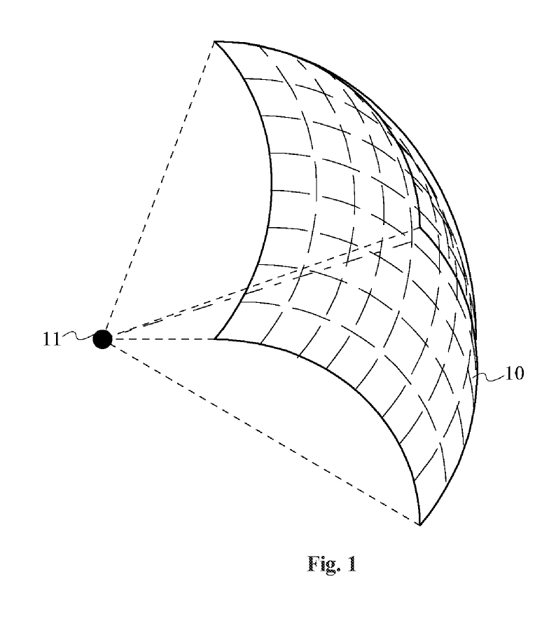

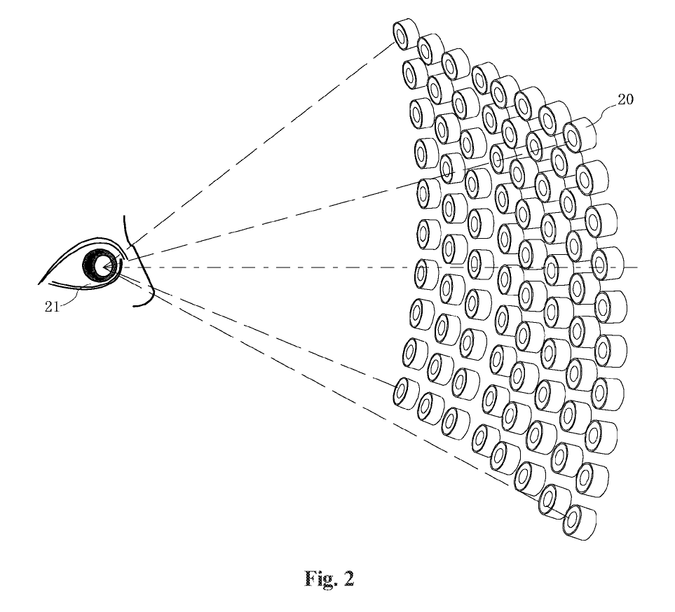

Desirably, the plurality of ultrasound generating elements are arranged upon a spherical surface that is focused upon the front surface of the subject eye. FIG. 1 is a schematic figure showing such a spherical surface. Lines orthogonal to the various portions of the spherical surface 10 all pass through the center point 11 of the sphere. And FIG. 2 is a schematic figure showing an example of how the ultrasound generating elements are arranged. By arranging the ultrasound generating elements 20 upon a spherical surface so that their outputs are pointed toward the interior of the sphere, their outputs are focused and all come together at the center portion of the sphere, and it is desirable to build the tonometer so that the subject eye 21 is positioned at this center portion. It is not necessary for this spherical surface to be a geometrically perfect sphere; it will be adequate for that surface to be sufficiently spherical for the outputs of the ultrasound generating elements 20 to be directed at the subject eye 21. With a structure of this type, the sound pressure of the sounds generated from the ultrasound generating elements 20 that come together at the position of the subject eye 21 becomes high. And the more it is possible to enhance the directivity of the sound pressure, the less the sound is human-audible at further than around 5 mm from the center of the sphere, for example.

While, in the preferred embodiment described above, the ultrasound generating elements 20 are arranged upon a spherical surface, as another preferred embodiment, it may also be suggested to dispose the ultrasound generating elements upon a plane, and to increase the sound pressure in the vicinity of the subject eyeball by adjusting the phases of the driving waveforms for the various generating elements by phase shifting these driving waveforms according to the distances of the elements from the center element.

With the tonometer of the present invention, due to the use of a parametric speaker, when the signals that drive the ultrasound generating elements are amplitude modulated at low frequency, due to the non-linearity generated by the sound, sound pressure is generated at low frequency (i.e. sound waves), but the sound pressure near the focus is further increased due to this low frequency sound pressure. And, by arranging the ultrasound generating elements in an appropriate configuration as described above, it is possible further to increase the directivity of the sound emitted, and thus to increase the sound pressure just in the neighborhood of the front surface of the subject eye. When vibrating the subject eye with this low frequency sound pressure, it is possible to generate sound waves only at the front surface of the subject eye, and neither the subject nor the people around the subject can hear this sound, so that it is possible to vibrate the subject eye without causing any feeling of discomfort to anybody. The subject eye has a characteristic frequency based upon causes such as its intraocular pressure and so on, and experiences vibration in response to the sound wave signal that it receives. Accordingly, a detection device that detects vibration data related to the subject eye is incorporated in the tonometer of the present invention as an essential structural element.

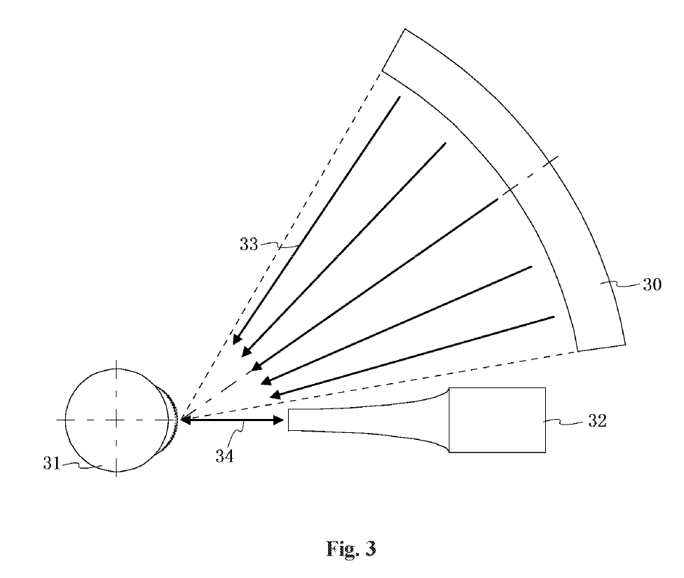

FIG. 3 is a diagrammatic figure showing an example of the tonometer of the present invention. Ultrasound waves 33 modulated at a low frequency are emitted from the parametric speaker 30, the subject eye 31 is vibrated by sound waves (i.e. by low frequency sound pressure) thereby generated in the neighborhood of the subject eye 31, and data 34 relating to these vibrations (i.e. to the amplitude thereof) is detected by an ultrasound sensor 32 that serves as a detection device.

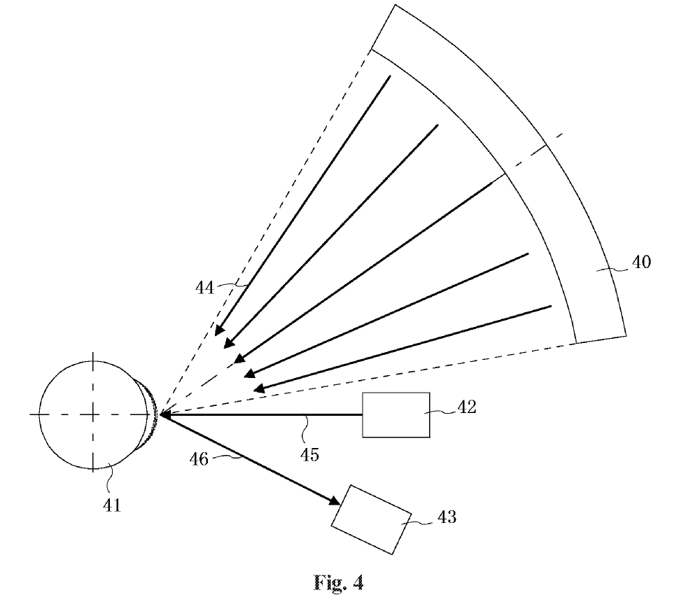

FIG. 4 is a schematic diagram showing another example of a tonometer according to the present invention. Ultrasound waves 44 modulated at a low frequency are emitted from a parametric speaker 40, the subject eye 41 is vibrated by sound waves (i.e. by low frequency sound pressure) thereby generated in the neighborhood of the subject eye 41, and data relating to these vibrations (i.e. to the amplitude thereof) is detected by an light detector 43 that serves as a detection device. In order to detect this vibration data for the subject eye 41 optically, for example, one possibility is that light for detection 45 from a light source 42 may be irradiated upon the subject eye 41, and that light 46 reflected form the subject eye 41 may be received by the light detector 43, or the like.

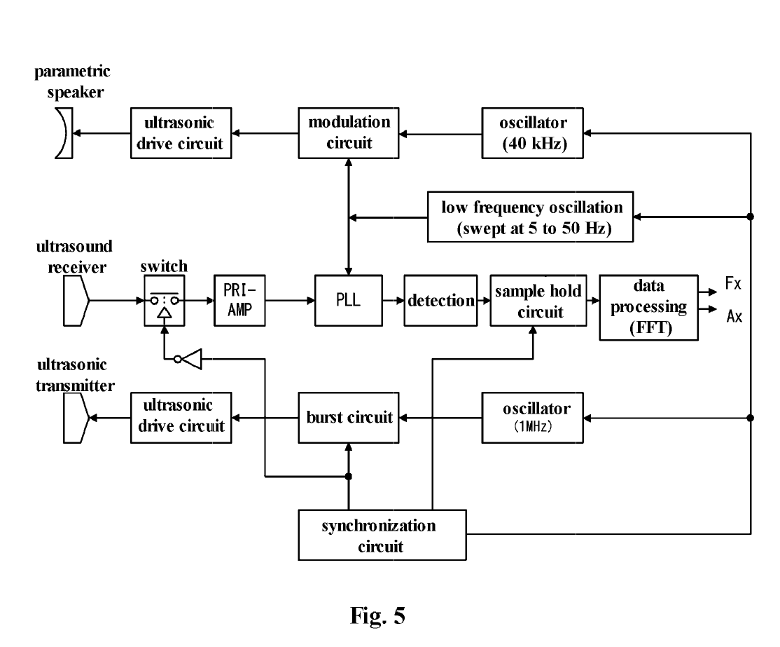

As described above, the method of detecting the vibration data for the subject eye is not particularly limited; an ultrasonic detection technique or an optical detection technique or the like may be employed, as appropriate. FIG. 5 is a schematic figure showing the structure of a tonometer according to the present invention. In FIG. 5, an example is shown in which vibration of the subject eye is detected by ultrasound. With this structure, vibration is detected by measuring advance and delay of the ultrasound propagation time caused by displacement of the front surface of the subject eye due to vibration. A signal at a frequency corresponding to ultrasound (for example, 40 kHz) is generated by an oscillator. And low frequency oscillations are superimposed at a frequency close to that of an audible frequency sound source (for example, 5 to 50 Hz). Modulation at this low frequency is applied to the ultrasound frequency signal, and the modulation is swept over time. The parametric speaker is driven at the modulated ultrasound frequency, and its generating elements, which are arranged upon a spherical surface, generate low frequency sound pressure (i.e. sound waves) only in the vicinity of the front surface of the subject eyeball. This low frequency sound pressure vibrates the subject eyeball, and, since this frequency is swept, the vibration of the eyeball increases at the point of resonance. In order to measure the amount of vibration (i.e. of displacement) of the subject eye, a higher frequency than the frequency at which the parametric speaker is driven is emitted from an ultrasonic transmitter, and is directed against the subject eye. Reflected ultrasound from the subject eye is captured by an ultrasound receiver and is detected, and change of the time for reflection is converted into an analog value. Since this change of the reflection time includes vibration of the subject eye (i.e. displacement of the front surface of the subject eyeball), accordingly the amount of vibration of the subject eye can be derived therefrom. The local maximum values of the frequency F and the amplitude A at this time (a plurality thereof) are recorded. The maximum values of the frequency Fx and the amplitude Ax among these recorded values are obtained, and these are taken as the vibration data for the subject eye and are supplied to subsequent processing for intraocular pressure calculation.

The intraocular pressure of the subject eye is calculated from the vibration data for the subject eye that has been detected as described above. The tonometer of the present invention includes a processing device that calculates the intraocular pressure of the subject eye from the detected vibration data for the subject eye. Fundamentally, this calculation of the intraocular pressure is processing that obtains the characteristic frequency of the eyeball of the subject eye from the vibration data that has been obtained as described above, and that derives the intraocular pressure, which is the internal pressure of the eyeball, from this characteristic frequency.

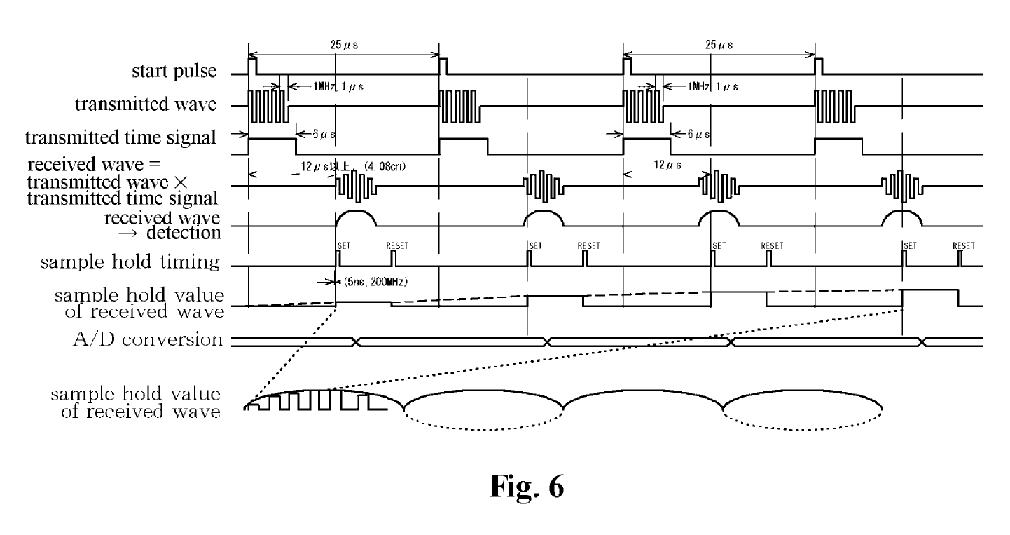

FIG. 6 is a schematic figure showing an example of signal timings employed in the present invention. The numerical values shown in this drawing are only specific examples, and are not to be considered as being limitative of the scope of the present invention in any way. The timings are shown of various signals when the vibration of the subject eye is being detected by ultrasound. The received returning reflected wave is detected, delay of this received wave causes change of the timing of sample hold, and change of the sample hold value reflects the vibration of the subject eye. Vibration of the subject eye is detected in this manner. Since the subject eye is vibrated at low frequency and the frequency of the applied vibration is swept, accordingly the amplitude of vibration increases when this frequency coincides with the resonant frequency of the subject eye. For example, it is possible to obtain the intraocular pressure value from a relationship between eyeball characteristic frequency and internal eyeball pressure that is created statistically. It should be understood that, in FIG. 6, vibration of the subject eye is shown as having been full wave rectified.

For correction of individual differences, it would also be possible to build the tonometer of the present invention so that it becomes possible to make data linkage with OCT, pachymeter, and/or ocular axis length measurement.

According to the present invention, it is possible to perform measurement in a perfectly non-contact manner without worrying about the noise being heard by the subject or the person doing the measurement or the like; the influence of vibration in the vicinity of the subject eye is minimized; it is possible to detect data relating to vibration of the subject eye at high accuracy; and accordingly it may be anticipated that intraocular pressure measurement can become simpler and more efficient.

What is claimed is:

1. A non contact eyeball vibration type tonometer, comprising:
a parametric speaker that directs sound waves against the front surface of a subject eye;
a detection device that detects vibration data of the subject eye caused by the sound waves from the aforementioned parametric speaker; and
a processing device that calculates the intraocular pressure of the subject eye from the aforementioned data of the subject eye.

2. The non contact eyeball vibration type tonometer according to claim 1, wherein the parametric speaker comprises a plurality of ultrasound generating elements that are arranged upon a spherical surface focused upon the front surface of the subject eye.

3. The non contact eyeball vibration type tonometer according to claim 1, wherein the parametric speaker comprises an ultrasound generating element capable of generating an ultrasound signal of 30 to 50 kHz that is modulated at a frequency of 10 to 100 Hz, and wherein said element is capable of sweeping said frequency over time.

4. The non contact eyeball vibration type tonometer according to claim 2, the aforementioned ultrasound generating elements being capable of generating an ultrasound signal of 30 to 50 kHz that is modulated at a frequency of 10 to 100 Hz, and capable of sweeping said frequency over time.

5. The non contact eyeball vibration type tonometer according to claim 1, wherein the detection device comprises an ultrasonic detection element.

6. The non contact eyeball vibration type tonometer according to claim 2, wherein the detection device comprises an ultrasonic detection element.

7. The non contact eyeball vibration type tonometer according to claim 4, wherein the detection device comprises an ultrasonic detection element.

8. The non contact eyeball vibration type tonometer according to claim 1, wherein the detection device comprises a light reception element that detects vibration of the subject eye optically.

9. The non contact eyeball vibration type tonometer according to claim 2, wherein the detection device comprises a light reception element that detects vibration of the subject eye optically.

10. The non contact eyeball vibration type tonometer according to claim 4, wherein the detection device comprises a light reception element that detects vibration of the subject eye optically.
